(19) United States
(12) Patent Application Publication
(10) Pub. No.: US 2008/0241065 A1
(43) Pub. Date: Oct. 2, 2008

(54) Systems and methods for the detection and analysis of in vivo circulating cells, entities, and nanobots

(76) Inventors: David A. Benaron, Portola Valley, CA (US); Illian H. Parachikov, Belmont, CA (US)

Correspondence Address: MORGAN, LEWIS & BOCKIUS, LLP., 2 PALO ALTO SQUARE, 3000 EL CAMINO REAL, PALO ALTO, CA, 94306, US

(21) Appl. No.: 11/729,395

(22) Filed: Mar. 27, 2007

(51) Int. Cl. A61B 5/145

(52) U.S. Cl. 424/9.1; 600/368; 356/39

(57) ABSTRACT

An improved circulating cell counter for generating light, and for delivering this light to a site in vivo for determining the presence, absence, concentration or count of a target cell, in which a light source such as a laser diode (121) and integrated optics (153) produce a beam transmitted to an in vivo target region (165), such as a capillary bed with flowing cells in a living tissue. Based upon the movement of cells in and out of this region, a circulating cell count (192) is generated, allowing determination of the presence, absence, concentration or count of the target cell. Use with optical, magnetic, or nanobot contrast agents, and methods of use are also described.

### U.S. Government Rights

The U.S. government has certain rights in this invention pursuant to Public Health Service contract CA105653 and CA107908, awarded by the National Cancer Institute to the Spectros Corporation.

### FIELD OF THE INVENTION

The present invention relates to detection systems and methods for providing highly specific cellular analysis of cells, entities, and/or xenograph nanobots in vivo, wherein the traditional ex vivo measurement is replaced by a measurement in living tissue. More particularly the present invention relates to systems and methods employing illuminating optics configured to illuminate and collect light from stained cells in the capillary circulation using a targeted optical dye, thus allowing for cell detection and/or counting in vivo and in real time, and allowing for on-line, real-time analysis of blood components without the need for blood withdrawal and preparation.

### BACKGROUND OF THE INVENTION

Blood cellular analysis (such as white cell counts, bacterial counts, T-cell counts, or circulating tumor cell counts) currently requires the withdrawal of blood, followed by laboratory microscopy, cell counting, flow sorting, or chemical/DNA/RNA/protein analysis. The collected fluids are often stained on slides, put through a cell counter, or probed using antigens and stains (for example, 1999 PNAS). Sometimes, the cells themselves are isolated and counted. By definition, all such systems require blood sample acquisition. Because of this, these methods are nearly universally restricted to ex vivo uses.

Not all types of cell analyses are amenable to blood sampling. For example, it is known that in patients with breast cancer there are rare circulating breast tissue cells. In breast cancer, this is about 1-5 cells per cc of circulating blood (compared with billions of red cells in the same cc of blood). In order to gather 10,000 tumor cells for analysis, one would need collect liters of blood. Such large blood sampling makes this method unacceptable for routine breast cancer diagnosis, or for serial testing to evaluate a response to treatment. Some have addressed this with magnetic sorting, antigens on tiny magnetic beads, to allow for enhancement of these rare cells prior to counting as described in U.S. Pat. No. 5,972,721 and published U.S. Patent Application No. 2006/024824, but the methods still requires obtaining blood samples each time the test is to be run.

Another example of tests that require blood drawing is the real-time analysis of infection. Patients in the intensive care unit, for example, frequently get widespread bacterial infection, a condition termed sepsis. Sepsis has a high mortality rate. Sepsis has certain markers, such as rising white blood cell count, rising fractions of certain white blood cell types, and rising levels of certain factors, such as IL-6, C-reactive protein, and the like, as well as rare circulating bacterial cells. To constantly monitor the blood for infection over time, liters of blood may again be required. This blood is then grown over time in a bacterial culture chamber after the blood has been removed from the body and placed in glass culture bottles as described in U.S. Pat. No. 5,356,815.

All of the above systems do not perform cell counts or they require blood or tissue sampling in order to perform circulating cell counts, and further are not designed for, and fail to reliably provide real-time analysis in living tissue without such a blood extraction.

None of the above systems suggest or teach a method and system for blood level analysis in vivo. Such an in vivo analysis has not been successfully commercialized to our knowledge. Accordingly, further developments are highly desirable and would constitute a significant advance in the art.

### SUMMARY OF THE INVENTION

The present invention relies upon knowledge of physiology, and of specific design considerations required to achieve in vivo cell counting.

A salient feature of the present invention is that cells move in vivo, creating a signal that can be analyzed, such as in capillaries with flowing blood.

Another feature of the present invention is that cells passing through a limited-field of detection, such as a narrow aperture optical fiber or a confocal apparatus, can produce detectable and countable “blips” on a single detector, or analyzable images on an imaging array, allowing for cell counting and/or analysis according to embodiments of the present invention.

Another salient feature is that, while the conventional systems require labeling of cells ex vivo, embodiments of the present invention provide that cells and markers can be labeled in vivo by injection, ingestion, or other means, allowing for enhanced specificity of in vivo cell counting and/or analysis.

Accordingly, in one aspect the present invention provides an in vivo noninvasive cell counting and analysis/or system.

Another aspect of the present invention is to provide specific cell labels via injection or ingestion of a contrast agent for improved specificity.

In some embodiments, the present invention relates to the coupling of a narrow aperture optical fiber or filter, set to illuminate and collect light from stained cells in capillary circulation using a targeted optical dye, thus allowing for cell counting in vivo and in real time, and allowing for on-line, real-time analysis of blood components without the need for blood withdrawal and preparation

Various embodiments of the present invention exhibit multiple advantages.

For example, one advantage is that screening procedures requiring large amounts of blood (such as rare cell screening) can be performed using an extended monitoring time, thus improving specificity and eliminating large blood draws.

Embodiments of the present invention additionally provide other advantages where in vivo circulating cell counting allows for real-time, continuous monitoring, thus allowing feedback to treatment, or for detection of an emerging process early in the course of the disease.

Further, circulating cells can be continuously monitored, such as in patients at risk for bacterial sepsis, providing an early warning system prior to the infection becoming difficult to treat.

Moreover, embodiments of the present invention provide for a flexible platform for testing of multiple assays, including white blood cell counts, differential cell counts, and the like.

There is provided a detector for use in performing in vivo circulating cell counting on living animals, with the option of specific cellular or chemical staining. In one example, an imaging system uses confocal lens system and spatial filtering for light collection at a specific plane of tissue, and an injected targeted dye, which can be assayed by the presence of non-random “blips” in intensity, signifying the passage of a labeled cell into the analysis area. The efficient detection allows this device to be deployed in the research lab, the clinical laboratory, or the Intensive Care Unit. Medical methods of use are described. Other configurations using magnetic beads and magnetic sensing are also described.

In one aspect, embodiments of the present invention provide a noninvasive in vivo circulating cell counting system, comprising: a detector functionally coupled to a target region and arranged to detect a signal within a living entity. The signal is representative of a contrast agent present in a target cell. A counter is provided which determines when a target cell passes through a field of view of the detector. Passage of the target cell is created by cell movement within said living entity. The counter may be configured to determine a number of parameters related to the target cell. For example, the counter may be configured to determine an estimate, measure, count, presence, absence, degree, or level of the circulating target cell.

In another aspect methods of monitoring a parameter related to the in vivo presence, absence, count, or concentration of a target cell type within a living entity are provided. Electromagnetic radiation is emitted into a target region of the entity, the emitted radiation is selected to interact with a reporter agent and/or target cells present in the living entity and moving through the region. A target signal returning from the region is detected over time or space; and the presence, absence, or concentration of the target cell in circulation in the living entity based upon a temporal change or distribution of the target signal within the region over time, is determined.

Additionally, some embodiments of the present invention provide an in vivo circulating cell counting system, comprising: a detector functionally coupled to a target region and arranged to detect a contrast signal within a living entity. The contrast signal is representative of a contrast agent present in one or more activated nanobots. A counter is provided that is configured to determine when an activated nanobot(s) passes through a field of view of the detector as it circulates within the living entity. The counter may be further configured to determine an estimate, measure, count, presence, absence, degree or level of the activated nanobot(s).

The breadth of uses and advantages of the present invention are best understood by example, and by a detailed explanation of the workings of a constructed apparatus, now in operation and tested in model systems and animals. These and other advantages of the invention will become apparent when viewed in light of the accompanying drawings, examples, and detailed description.

### BRIEF DESCRIPTION OF THE DRAWINGS

Advantages and embodiments of the present invention will become apparent upon reading the following detailed description and upon reference to the following figures, in which:

FIG. 1 is a schematic diagram of a system constructed in accordance with some embodiments of the present invention;

FIG. 2 shows model data from the system of FIG. 1;

FIG. 3 shows a display of results from the data of FIG. 2;

FIG. 4 shows a system constructed in accordance with other embodiments of the present invention based upon a commercial confocal endoscope; and

FIG. 5 shows a circulating nanobot that becomes fluorescent upon binding to a bacteria in accordance with some embodiments of the present invention.

### DETAILED DESCRIPTION OF THE INVENTION

### Definitions

For the purposes of this invention, the following definitions are provided. These definitions are intended for illustration purposes only, and are not intended to limit the scope of the invention or appended claims in any way.

Real-Time: A measurement performed in an ongoing manner, or within a few minutes. In medical or surgical use, such real-time measurements allow a procedure or a treatment plan to be modified based upon the results of the measurement.

In Vivo: A measurement performed on cells on or within a living animal, plant, viral, or bacterial subject. A living animal includes all mammals, including humans.

Tissue: Sample material from a living animal, plant, viral, or bacterial subject, with an emphasis on mammals, especially humans.

Target Cell: Cell type or types for which analysis is desired.

Target Region: A physical region at which a sample or tissue to be analyzed is to be placed. The target region in an optical system is the area illuminated and monitored by a detector.

Target Signal: An optical signal specific to the target cell. This signal may be enhanced through use of a contrast agent. This signal may be produced by scattering, absorbance, phosphorescence, fluorescence, Raman effects, or other known spectroscopy techniques.

Reporter or Contrast Agent: A molecule or material (such as an iron ferrite bead, a dye, a quantum dot, or a light scatterer) that creates a detectable signal. This signal may be created or change when it interacts with a target cell (or substance in, near, or around a target site), such as a unblocking of photoquenching during proteolysis of a closely-paired but protease-site linked cyanine dye, or for a color-shifting dye in response to pH. This optical signal is detected by the optical detector, often but not always in response to an optical illumination. The illumination could in practice be via non-optical means, such as a radiowave or magnetic field, or a luciferase based molecule could be used, which generates light in response to energy consumed at the cellular level.

Nanobot: A small, self-contained cell-like object that can circulate and perform functions. One function could be a reporting function, such as changing its fluorescence, polarization, magnetism, light scattering, or Raman cross-section in response to conditions or entities within a living entity. Then, these conditions could be estimated, counted, detected, or measured by an external detector that detects the nanobot's signal. Further, the nanobot could additionally be constructed to perform a therapeutic function, in response to an internal or external signal or power source, once the condition has been detected or localized. In some embodiments a nanobot may be defined as a nanomachine, sometimes referred to as a nanite, which is a mechanical or electromechanical device whose physical dimensions, or key functioning element dimensions (such as an engineered optical receptor) are measured in nanometers.

Scattering Material: Material that scatters light as a significant feature of the transport of photons through the sample. Most tissues in vivo are scattering materials.

Light: Electromagnetic radiation from ultraviolet to infrared, namely with wavelengths between 10 nm and 100 microns, but especially those wavelengths between 200 nm and 2 microns, and more particularly those wavelengths between 450 and 650 nm.

Light Source: A source of illuminating photons. It may be composed of a simple light bulb, a laser, a flash lamp, an LED, a white LED, or another light source or combination of sources, or it may be a complex form including, a light emitter such as a bulb or light emitting diode, one or more filter elements, a transmission element such as an integrated optical fiber, a guidance element such as a reflective prism or internal lens, and other elements intended to enhance the optical coupling of the light from the source to the tissue or sample under study. The light may be generated using electrical input (such as with an LED), optical input (such as a fluorescent dye in a fiber responding to light), or any other source of energy, internal or external to the source. The light source may be continuously on, pulsed, or even analyzed as time-, frequency-, or spatially-resolved. The light emitter may consist of a single or multiple light emitting elements, such as a combination of different light emitting diodes to produce a spectrum of light. An optical reporter is optically coupled to a light source if light from the source reaches the dye. Other electromagnetic sources may be used, such as magnets for use in detecting ferrite-based contrast agents.

Light Detector: A detector that generates a measurable signal in response to the light incident on the detector. In this system, the detector is variably a photodetector or a CCD imaging chip, though other detectors could be substituted by one skilled in the art. An optical reported optically coupled to a detector if the detector receives light that his been influenced or interacted-with by the dye. Other detectors that do not detect light may also be used, such as magnetic field detectors (e.g., SQUID).

Optical Coupling: The arrangement of two elements such that light exiting the first element interacts, at least in part, with the second optical element. This may be free-space (unaided) transmission through air or space, or may require use of intervening optical elements such as lenses, filters, fused fiber expanders, collimators, concentrators, collectors, optical fibers, prisms, mirrors, or mirrored surfaces. For example, a dye is optically coupled to an illuminator if the light from the illuminator reaches the dye, while a dye is optically coupled to a detector if the detector receives light that his been influenced or interacted-with by the dye.

One embodiment of the device will now be described. This device has been designed and numerically evaluated in the laboratory in experimental tests, under support from the U.S. Government. Data from such tests are included in some of the examples that follow the initial description of one embodiment of the system.

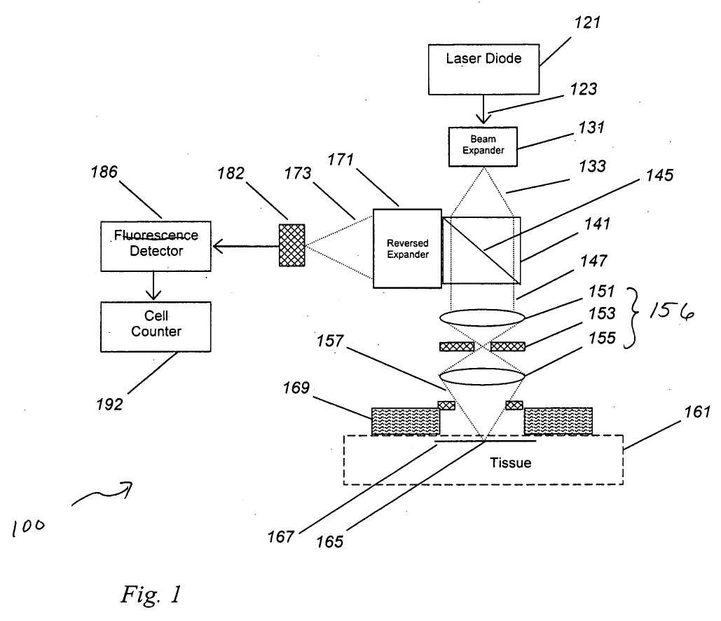

In the system shown FIG. 1, an exemplary system for analyzing in vivo circulating cells is illustrated in its component parts. Generally, in some embodiments the system is comprised broadly of a light emitter, a light detector and a cell counter. The light emitter may be comprised of Laser diode 121 which generates illumination light ray 123. In this case, the detection is optical rather than magnetic. Ray 123 enters beam expander 131 to create expanded beam 133. Beam 133 enters dichroic beam splitter 141, which contains dichroic element 145. Beam 133 is unaffected by splitter 141 and dichroic 145, and emerges as beam 147. The purpose of dichroic element 145 becomes important only later, when it deflects the light returning back from the other direction, as will be explained below. In some embodiments, Beam 147 enters a triplet of condensing lens 151, pinhole 153, and expanding lens 155, all of which constitute a spatial filter 156 to reject out-of-plane light on the return path, and therefore do not substantially affect the illumination light with emerges as beam 157, which is focused on target site 165. Other filter arrangements, including non-confocal designs, fall within the spirit of the invention if they are used for measuring, detecting, or counting circulating cells, entities, or nanobots within a living body.

In this example, target site 165 is located under the surface of tissue 161 in a living entity (entity not shown). However, it is important to note that tissue 161 nor the entity is a component part of this invention; thus tissue 161 is shown as a dashed-outline box to indicate that it is not a part of the invention. Tissue 161 is shown only to illustrate use of the device. Target site 165 exists on focal plane 167, which is where the light is focused. Some of this light interacts with the target region, such as cells with dye located at target site 165.

Light leaving target region 165 has interacted with nearby light-scattering tissue 161, with target cells at the target region 165, and with any contrast agent (not shown) that may or not be associated with any target cells (not shown) that may be at or near target region 165.

Light that has interacted with region 165 now disburses (scatters, travels, fluoresces, is generated, or otherwise travels away from region 165) in many directions. Some of this light (which can be in a random process considered to be the fractional area of the surface of a diffuse light sphere traveling in all directions) travels back along the identical path of beam 157, only now in the opposite direction. This light behaves as if it came from target region 165, and passes through the spatial filter composed of lenses 155 and 151 and pinhole 153. Continuing backwards along the same path as beam 147, the returning light strikes dichroic element 145. Because in this case the returning light has a different wavelength than the emitted light, whether due to fluorescence, wavelength shift, interaction with a quantum dot, or other process, the returning light does not pass through dichroic element 145, but instead reflects into reversed expander 171. Expander 171 focuses returning light as beam 173, a new path not taken by illuminating light, into notch filter 182. Notch 182 removes any residual light from the initial illumination, and passes it to detector 186. Detector 186 may be comprised of any suitable detector, such as but not limited to a single element, such as an avalanche photodiode or photomultiplier tube, a wavelength-resolved CCD, or it may even be an imaging device that makes a planar image returning light from target region 165.

Based upon the signal(s) from detector 186, cell counter 192 now determines the presence, absence, speed, concentration, or other feature of the target cell. For example, cell counter 192 may use the total amount of hemoglobin seen to estimate a volume of capillaries being measured and then use the number of tumor cells seen over time to estimate a concentration of tumor cells per cc of blood. Alternatively, cell counter 192 may use multiple dye reporters to discriminate between gram positive and gram negative bacteria, to give a signal as to the presence of each. A threshold may be used to set a diagnosis, such as impending or existing sepsis, in this analysis. Last, multiple features may be used in order to make a more complex diagnosis using multiple dyes, T- and B- white cell subtype levels, or the presence of activated macrophages in the circulating blood. The programming of such counters falls within the ordinary skill of those skilled in the art, and is known in the art for use in ex vivo flowing cell benchtop equipment.

Optionally, the relative size and depth of the spot size of region 165 can be adjustable using lenses such as the lens/pinhole spatial filter described earlier.

Returning light, returning from the focal point after interaction with material at region 165, can also transmit information either temporally (such as signals from appearing/disappearing flowing cells), or spatially (such as images of moving spots from cells imaged in capillaries), or both.

In order to achieve a limited field of view, different methods can be used. In this example, the spatial filter and confocal geometry serves as a spatial filter, but this could be merely instead a small optical fiber replacing lenses 151 and 155 and filter 153 with a limited depth of field fiber, as determined by the wavelength used.

Optionally, a destructive element, such as laser 121, can be amplified, or a new laser added, to optionally destroy cells based on thermal, absorbance, or other properties, making this system potentially therapeutic.

There are many such devices that could be adapted to perform this function guided by the teaching of the present invention, such as a confocal endoscope or a confocal imager with a CCD attachment, thus allowing image analysis to pick out the flowing cells from the static, nonmoving background. Commercial confocal microscope/endoscopes are known, such as from Mauna Kea (Cambridge, Mass.), and modification of each of these is within the grasp of a well-informed person skilled in the art, and are therefore incorporated into this disclosure by reference.

Methods of operation of the device may now be described.

After injection of a targeted dye, the dye achieves a non-uniform distribution of contrast agent signal. This may mean, for example, that the dye merely binds to cells, some of which have already entered the circulation or will enter the circulation. The assembly of sufficient dye on the cell then creates a large “blip” of contrast as these labeled cells flow through the detection field. Here, those cells which are labeled with a concentration of dye above the background produce a “blip” in the detected intensity signal. Each blip is considered a count. In this case, if the volume of blood measured, the signal is corrected for this blood volume using a spectrophotometric analysis of total hemoglobin, while the transit time is corrected for using the length of the blip.

However, the dye does not merely need to label circulating cells. The reaction could require that cells with dye undergo an activation step by coming into contact with another cell, protein, pH, or other environmental signal. Further, one can inject a micelle containing dye that is “activated” by the presence of the target cell, thus producing a contrast-micelle that is detected during passage under the detector. This should make it clear that it is not merely the labeling of cells by dye while flowing, but a labeling of cells or objects that may at one time or another be induced to flow under the detector. This is critical as many cell types (white blood cells, stem cells) spend only a small fraction of their lives actually circulating.

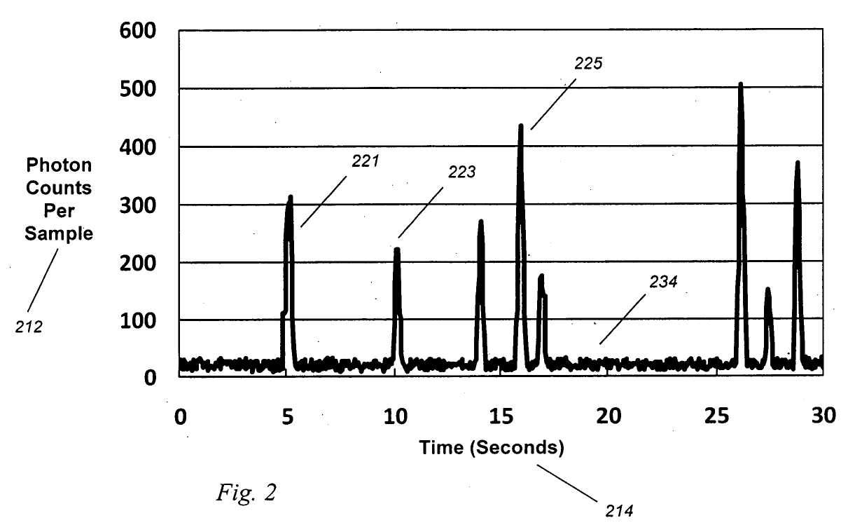

Data from a sample of injected cells flowing under a detector are shown in FIG. 2. Here, photon counts per sample 212 are plotted over time 214. Highly peaked, short lived blips, such as blips 221, 223, and 225, are seen when a cell passes or flows under the system. In between cell detection, background 234 varies with pulsations of the heart, changes in confocal coupling to the tissue, and random fluctuations

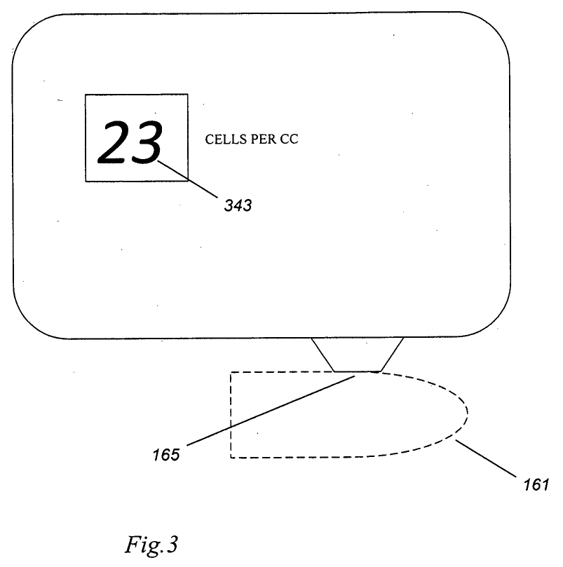

A concentration of the detected cells is shown in FIG. 3, where the value is displayed on display 343 while measuring on tissue 161 Again, tissue 161 per se is not considered a part of this invention, and is shown for illustrative purposes only. Rather, site 165 merely needs to be located at a site in which the flow of blood in a living organism generates the temporal and spatial changes in optical signal that allow for cell counting.

Of note, when light from a noninvasive or invasive system penetrates into tissue, the photons traveling between the light source and the light detector take a wide range of paths. The present device takes advantage of this effect as the scattering provides an averaging and volume sampling function. When detected illumination is measured after it has propagated through the tissue over substantially non-parallel multiple courses taken through the tissue between the time the photons are emitted and then detected, many regions of the tissue can be sampled, not merely the tissue on a narrow line between emission and detection. This allows a small but important feature, such as a the ability to sample the subsurface capillary layer of a fingernail capillary bed, even if the probe itself is placed on the outer surface of the nail.

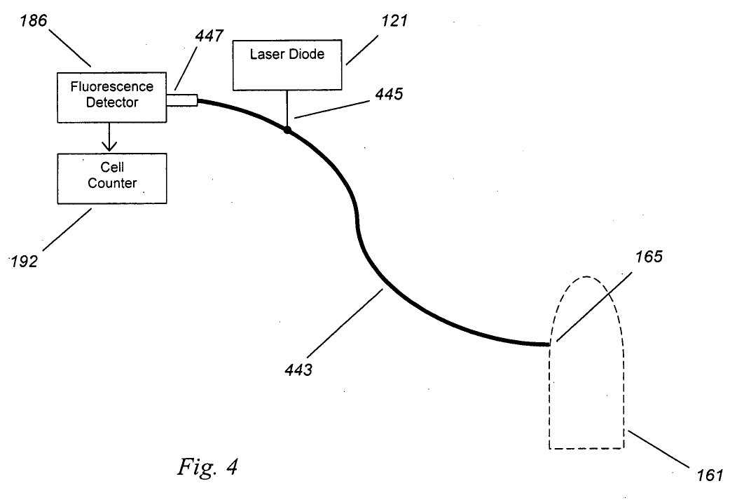

FIG. 4 illustrates another embodiment of the present invention where the system employs a confocal imaging device. Confocal imaging devices are commercially available. In some embodiments, for example, referring to FIG. 4, confocal endoscope 443 is coupled to light source 121 through fiber 445, and photodetector 186 and cell counter 192 are connected directly to eyepiece 447.

In this embodiment, a light source is laser diode 121. Alternatively, a light source may be comprised of a broadband LED, a narrow line LED, a white light bulb, a polymer plastic that emits light under the influence of electrical power, or be a laser with broadening of the waveband via the input fiber impregnated with fluorescent dye, and so on, provided only that the light source meets the technical requirements of the system disclosed herein.

### EXAMPLES

The breadth of uses of the present invention is best understood by example, seven of which are provided below. These examples are by no means intended to be inclusive of all uses and applications of the apparatus, merely to serve as case studies by which a person, skilled in the art, can better appreciate the methods of utilizing, and the scope of, such a device.

### Example 1

### Expected Lower Limit of Cell Detection

We estimated the minimum number of cells detectable.

In many (but not all) cases, the detected cells are in the vascular compartment, which provides the flow needed to generate the cell counting signals. The vascular volume (e.g., the volume of the tissue measured that resides within the vascular compartment) was estimated and then verified by experiment. As an estimate, human tissue has an average blood volume of about 2%, but this can be as high as 10% or more when imaging a capillary-rich bed. With a view of 1 mm and a wide focusing depth of 1 mm, this yields a tissue measurement volume of 1 uL, or a vascular volume of 0.1 uL or less. This level of vascular contact has been confirmed in laboratory tests, with volumes as high as 100 uL for large fiber probes, and volumes under 1 uL for the smallest probes.

Next, the concentration and count of the target cell was estimated. Cell counts for normal blood elements are known. For white blood cells (WBC's) the normal concentrations are 4,000-10,000 cells/uL; for “band forms” seen in infection, this is typically 1-3% of WBC's and can rise to 25% or more during infection. At the volume of the probes described above, this would yield a normal count of less than 1 to over 125 cells per field at any given moment.

The concentration of more rare cells can be estimated, such as for breast cancer cells circulating from solid tumor, simply by increasing the measurement volume, or by increasing the time required. For example, in normal subjects without cancer, 800 cells are of epithelial origin per liter of blood, while in cancer this rises to 6,100 per liter. For a probe with a 1 uL measurement volume, one would see a tumor cell every 3 minutes with 10 cells/cc. Therefore, in order to generate a statistically valid measure, a 30 minute measurement time would be required. For the largest measured volume, this would be 0.7 cancer cells per field, with each cell requiring 6 seconds to pass through the detector (at 1-2 mm/sec transit time for cells); for the smallest measured volume, this would be 6×10⁻⁶ cells per field, with an average transit time of 0.1 sec and a time between cells of 16,000 seconds. Based upon this, an ideal sampling volume might be about 2 uL, yielding 1 cell per 20 seconds, on average.

As noted, this time can be decreased through the use of larger area measures (limited only by signal to noise, which decreases at increasing volumes of measurement), or through parallel sensors, such as 100 sites measured simultaneously, which would reduce the measurement time 100-fold.

There are also ways to increase the flow rate. For example, the fingertip can be warmed, or alternatively the blood volume can be expunged and returned via pressure and release to get more rapid flow locally during capillary refill, and thus larger changes for more rapid detection of low-prevalence cells.

It will be obvious to one skilled in the art that other measures of flow can be added to provide additional information. For example, Ultrasound can be used to monitor local flow rates, and used to adjust the cell counts according to local flow rates.

The level of signal generated by a single cell is now estimated. With an illumination of 1 W/cm², this yields about 650,000 photons hitting each cell per second. If we assume a field-effect region for each dye molecule is 100 nm, then 65 photons strike each dye molecule per second. With 100,000 dye molecules on the surface of each cell to be detected, and a quantum efficiency of 0.2, this would produce 1.6 million photons/second from each cell. Of course, such cells would bleach during illumination, but each cell is likely only measured once. Assuming we are measuring 2 millimeters away, and that we capture light using a 200 micron fiber, we should see 8,000 photons per second, versus a background of 20 photons per second. This is well into the detectability range for tumor cells or bacteria. We assume that the cell label circulates for hours, but that it does not clump or self-associate.

Optionally, multiple stains can be used simultaneously, such that a cell is only counted if both markers increase or decrease simultaneously.

### Example 2

### Detection a Model of Tissue

In order to test the validity of the data generated using the model shown in Example 1, we constructed a working system and tested this in a fluid model of tissue.

We have shown that in vivo circulating cell counting is feasible. Such improved lens systems may be designed as a standalone device, or embedded into a diagnostic or therapeutic system.

We have discovered an improved circulating cell counter that operates in vivo. A fiber-based illumination and detection system as been constructed and tested, in which a fiber optic system is used for light collection and collection, and a photodetector has been used to detect and quantify “blips” in returning light. A medical system incorporating the improved device, and medical methods of use, are described. This device has been built and tested in several configurations in models, animals, and planned for humans, and has immediate application to several important problems, both medical and industrial, and thus constitutes an important advance in the art.

### Example 3

### Detection of Circulating Prostate Tumor

By creating a ligand targeted against the extracellular domain of PSMA, a molecule found on the membrane of cells in duct tissue in the prostate gland, one has a binding target that is found only on the surface of prostate cells, and to a lesser extent on new blood vessels (neoangiogenesis). This binding site is also found on circulating tumor cells, such as in prostate cancer.

We created a ligand using the hj-591 antibody developed by Bander et al. at Cornell University, and coupled this to CyDye (Amersham Health, General Electric, England) using chemistry pathways under the direction of Darryl Bornhop at Vanderbilt University. This work was funded by the US Government (PHS Grant CA107908, David Benaron, Principal Investigator).

Because the dye binds to prostate cells, circulating tumor cells may be detected using the methods and systems described in Examples 1 and 2.

### Example 4

### Detection of Circulating Ovarian Cancer Cells

By creating a ligand targeted against the folate receptor of ovarian cancer cells, a molecule found on the membrane of many cells but up-regulated 400-fold in cells that are cancerous, one again has a binding target that is in the circulating blood only on the surface of ovarian tumor cells, and to a lesser extent can be found on activated, circulating macrophages.

A folate receptor (FR) agent was developed for this purpose under work funded by the US Government (PHS Grant CA105653, written 2002-2003, David Benaron, Principal Investigator). Extensions of this work by our group and others will be published by Low and others.

### Example 5

### Detection of Circulating Bacteria Using Dyes or Nanobots

Circulating bacteria are present in patients well before the circulating infection (called bacteremia, or bacteria in the blood) becomes clinically significant. Once the infection is well developed, patients are at high risk for injury or death, and sepsis remains a major killer. It is estimated that 1,000,000 people a year die from sepsis in the United States.

A binding agent can be developed against certain agents present on the surface of bacteria. Some of these agents are specific to a particular bacteria, while others may be against a group or family of bacteria. If a dye is injected, and a bacteria is present, this dye will accumulate on the surface of the bacteria, making the bacteria detectable in the same manner that a circulating tumor cell is detectable.

It is worth noting that the number of binding agents has increased enormously, including agents that bind to surface proteins, to intracellular proteins and mRNA, and even to nuclear binding agents specific to DNA strands (in some primitive cell types, the prokaryotes, the DNA is free in the cell as there is no nucleus or nuclear membrane). These agents may activate and become fluorescent upon binding, or they may split or change wavelength upon the presence of a particular protein or molecule. All of these binding and signaling processes are within the skill of a well-informed person, and are incorporated into this disclosure.

Further, various molecules can give their signaling by fluorescence, luminescence, phosphorescence, optical scatter, optical rotation, polarization, and other optical signaling means. Again, each of these fall within the spirit of the instant invention.

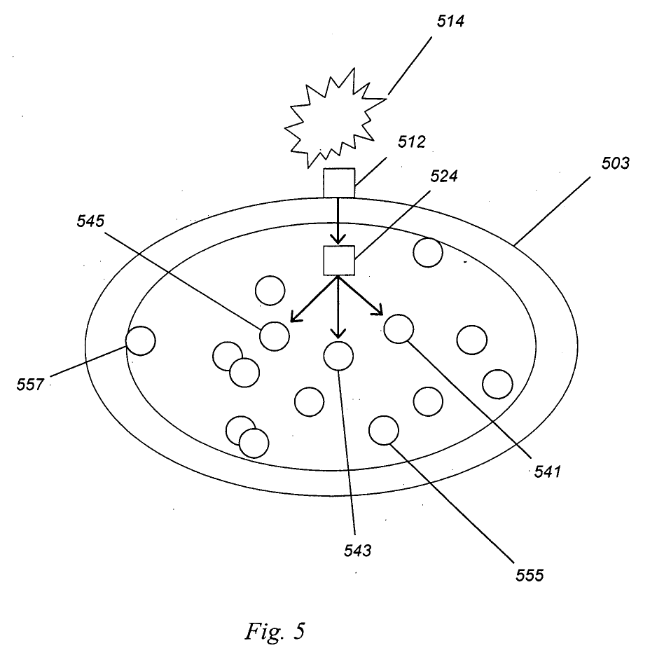

In some embodiments a small, self-contained cell-like object that can circulate and perform function(s), called a nanobot, may also be reasonably employed to generate a signal in response to the presence of a bacteria or other condition. Referring to FIG. 5, in the exemplary embodiment nanobot 503, a nanite, is a small xenograft (a foreign tissue inserted into a living host). Nanobot 503 is configured to be sensitive at binding site 512 to the presence of bacteria 514 (or tumor cell, or any other agent, compound, molecule, or entity). For example, when bacterial 514 binds to site 512, the nanobot could contain machinery to release fluorescent molecules contained in trapping cage 524, and free them into the interior of nanobot 503, as shown as freshly-released fluorescent molecules 541, 543, and 545, and by far-diffused released molecules 555 and 557. Molecules in cage 524 are held close together, which produces a phenomenon known as quenching that greatly reduces or eliminates fluorescence. However, once molecules 541, 543, 545, 555, 557, and thousands of other released molecules (not shown for clarity) diffuse into the nanobot, these molecules become fluorescent, thus producing fluorescence through a non-genetic chemical process in response to the binding of Staph. aureus, a bacteria of interest that could be the target cell to be counted, onto the outer surface of nanobot 503. This is an amplification, in which a single binding event produces a larger signal. Alternatively, and fully within the spirit of the invention, the nanobot could produce a signal via changes in polarization, magnetism, light scattering, Raman cross-section, or any other externally detectable, countable, or measurable response to conditions or entities within a living entity.

Once a signal is created, these activated nanobots could then be estimated, counted, detected, or measured by an external detector that detect the nanobots' signal. Further, the nanobot could additionally be constructed to perform a therapeutic function, in response to an internal or external signal or power source, once the condition has been detected or localized.

While the invention has been described in connection with specific embodiments it is evident that many variations, substitutions, alternatives and modifications will be apparent to those skilled in the art in light of the foregoing description and teaching. Accordingly, this description is intended to encompass all such variations, substitutions, alternatives and modifications as fall within the spirit of the appended claims.

What is claimed:

1. A noninvasive in vivo circulating cell counting system, comprising:
a detector, said detector functionally coupled to a target region and further arranged to detect a signal within a living entity, said contrast signal representative of a contrast agent present in a target cell; and
a counter, which determines when a target cell passes through a field of view of said detector, said target cell passage created by a cell movement within said living entity, for determining a target cell estimate, measure, count, presence, absence, degree, or level.

2. The system of claim 1, wherein said contrast agent is a ferrite bead, and said detector is comprised of a magnetic field detector.

3. The system of claim 1, wherein said contrast agent is an optical contrast agent, said detector is comprised of a photodetector, and said system further comprises a light source, said light source optically coupled to said target region.

4. The system of claim 1, wherein said contrast agent is targeted to circulating bacteria.

5. The system of claim 1, wherein said contrast agent is targeted to ovarian cancer using a folate receptor.

6. The system of claim 1, wherein said contrast agent is targeted to prostate cancer using a PSMA extracellular membrane protein.

7. The system of claim 1, wherein said contrast agent is located in an injected and circulating micelle, said micelle operating as said target cell, and further wherein said contrast signal is induced in said micelle by contact with a selected cell type, protein, pH, or other trigger.

8. A method of noninvasively monitoring a parameter related to the in vivo presence, absence, count, or concentration of a target cell type within a living entity, comprising the steps of:
emitting electromagnetic radiation into a target region of the entity, the emitted radiation selected to interact with a reporter agent and/or target cells present in the living entity and moving through the region;
detecting over time or space a target signal returning from said region; and
determining a parameter related to the presence, absence, or concentration of the target cell in circulation within the living entity based upon a temporal change or distribution of the target signal within the region over time.

9. A noninvasive in vivo circulating cell counting system, comprising:
a light emitter, said emitter optically coupled to a target region located within a living entity;
a light detector, said detector optically coupled to the target region and further arranged to detect light from said emitter after having interacted with said target region; and
a counter, configured to determine when a target cell has passed into or out of a field of view of said detector, said target cell motion created by a cell movement within said living entity.

10. A method of noninvasively monitoring the in vivo presence, absence, count, or concentration of a target cell type within a living entity, involving:
providing an optical contrast reporter agent;
allowing time as required for the reporter to achieve a distribution within the living entity and to interact with the target cell type;
emitting light into a target region of the entity, the light selected to interact with the reporter and/or target cells moving through the region;
detecting over time or space a target light signal returning from returning from the target region as a result of the interaction of the emitted light with the contrast agent; and
determining the presence, absence, or concentration of the target cell based upon a temporal change or distribution of the target signal in circulation within the region of the living entity over time.

11. An in vivo circulating cell counting system, comprising:
a detector, said detector functionally coupled to a target region and further arranged to detect a contrast signal within a living entity, said contrast signal representative of a contrast agent present in one or more activated nanobots and
a counter, configured to determine when an activated nanobot passes through a field of view of said detector, said nanobot passage created by a cell movement within said living entity.

12. The system of claim 1 wherein said counter is further configured to determine an estimate, measure, count, presence, absence, degree or level of an activated nanobot.

13. The system of claim 9 wherein said counter is further configured to determine an estimate, measure, count, presence, absence, degree or level of an activated nanobot.

14. The system of claim 11 wherein said counter is further configured to determine an estimate, measure, count, presence, absence, degree or level of the activated nanobot.
